(19) United States
(12) Patent Application Publication
(10) Pub. No.: US 2009/0143452 A1
(43) Pub. Date: Jun. 4, 2009

(54) Use of prochloraz for wood protection

(76) Inventors: Rainer Bruns, Leverkusen (DE); Martin Kugler, Leichlingen (DE); Thomas Jaetsch, Koln (DE)

Correspondence Address: LANXESS CORPORATION, 111 RIDC PARK WEST DRIVE, PITTSBURGH, PA, 15275-1112, US

(21) Appl. No.: 11/920,206

(22) PCT Filed: May 4, 2006
(86) PCT No.: PCT/EP2006/004159

§ 371 Date: Dec. 3, 2008

(30) Foreign Application Priority Data
May 13, 2005 (DE) 10 2005 022 149.1

(51) Int. Cl. A01N 43/50; A01P 3/00; C07D 233/90

(52) U.S. Cl. 514/399; 548/333.5

(57) ABSTRACT

The active compound prochloraz and fungicidal compositions based on prochloraz are highly suitable for protecting wood and timber products against attack and/or destruction by soft-rot fungi.

The present invention relates to novel fungicidal compositions based on the active compound prochloraz and to the use of prochloraz and of fungicidal compositions comprising prochloraz for protecting wood and timber products against attack and/or destruction by soft-rot fungi.

In principle, the person skilled in the art distinguishes between structural destruction of wood by Basidiomycetes and—if the wood is exposed to an environment with high humidity or soil contact—by soft-rot fungi, and an optical impairment of the wood by wood-discolouring fungi.

Active compounds for protecting wood against destruction by fungi have been known for a long time; thus, for example, tar oils have been used for protecting wood. Tar oils have good permeability and high resistance to leaching; however, owing to their persistency, their odour and their toxicologically unfavourable properties they have clear disadvantages, so that nowadays their use in the protection of wood is very limited.

As an alternative for the permanent protection of wood, active compounds based on chromium, copper and arsenic have been used; however, owing to their toxicological and in particular ecotoxicological disadvantages, they have come under pressure to be substituted.

On this background, modern compositions for the protection of wood based on organically active compounds have been developed. Triazole fungicides, such as tebuconazole or propiconazole, may be mentioned by way of example.

However, in practical applications, it is found that such modern organic active compounds are not sufficiently effective for protecting wood in permanent contact with soil or in environments with high humidity, i.e. in risk class 4, and have an activity gap against soft-rot fungi, so that for these applications, the active compounds described at the outset, such as tebuconazole or propiconazole, have to be combined with metals or metal salts, in particular copper salts. Furthermore, in compositions for protecting wood for this risk class, use is still made of inorganic agents for protecting wood, such as boron compounds, silicofluorides, chromium- and fluorine-containing salts, chromium- and copper-containing salts with and without arsenic, chromium- and copper-containing salts with and without boron compounds and also betaine preparations based on polymeric betaines in combination with boron and copper salts, and also highly water-soluble quaternary ammonium compounds.

From an ecological point of view, the use of compositions for the protection of wood which comprise heavy metals is to be considered to be objectionable. Accordingly, there is still a demand for improved compositions for the protection of wood, which compositions are based on active compounds which provide a satisfactory protection of wood without heavy metals being added.

To date, in the protection of wood, the fungicides used against attack by Basidiomycetes are triazole fungicides, such as, for example, tebuconazole, propiconazole or cyproconazole. Also employed are the fungicides IPBC, cabendazim, folpet and dichlofluanid for protection against wood-discolouring fungi. However, in the test against soft-rot fungi according to ENV 807, both the triazoles and the fungicides mentioned with action against blueing fungi fail entirely or are insufficiently active.

Prochloraz (N-propyl-N-[2-(2,4,6-trichlorophenoxy)ethyl]imidazole-1-carboxamide; CAS No. 67747-09-5) is a known crop protection fungicide having a relatively broad activity spectrum.

By virtue of the action of prochloraz against Ascomycetes and Deuteromycetes in crop protection, it has, on occasions (cf., for example EP-A 1025967), been proposed to employ prochloraz for protection against wood-discolouring fungi; however, in practice, the effectiveness has not been confirmed. In contrast, it is known that prochloraz is insufficiently active against blueing fungi (cf. J. A. Drysdale et al., New Zealand Journal of Forestry Science 1982, 12 (3), 457-466).

Furthermore, it has been described that mixtures of prochloraz with tri-n-butyltin compounds are suitable for protecting wood and industrial materials against attack by microorganisms (cf. DE-A 3522788). Furthermore, it is known that prochloraz can be used in combination with NOIT (N-octylisothiazolinone) for protection against wood-discolouring fungi (cf. NZ-A 331830). In both cases, based on the described unsatisfactory activity of prochloraz in the protection of wood, it has to be assumed that the action claimed is due essentially to the respective mixing partners.

Surprisingly, and entirely unexpectedly, it has now been found that, in the test according to ENV 807 (European Norm Voluntary) against soft-rot fungi, prochloraz has excellent activity, whereas frequently used fungicides against bluestain, such as IPBC and imazalil, show, if any, only an entirely insufficient activity against soft-rot fungi in the test according to ENV 807.

Accordingly, the present invention provides the use of prochloraz (N-propyl-N-[2-(2,4,6-tri-chlorophenoxy)ethyl]imidazole-1-carboxamide) for protecting wood, timber products and wood/plastic composites against attack and/or destruction by soft-rot fungi.

The use according to the invention is a substantial and decisive addition to the prior art, since it is now possible to substitute ecotoxicologically objectionable heavy-metal-comprising wood protection compositions for protecting wood in permanent contact with soil or in environments with high humidity, i.e., for example, in risk class 4. This reduces the impact on the environment by heavy metals considerably.

Prochloraz displays a broad activity against soft-rot fungi, such as, for example, against the fungi described in ENV 807, such as Chaetomium globosum, Glenospora graphii, Humicola gisea, Petriella setifera, Trichurus spiralis and Lecythophora mutabilis, and also against Trichoderma viride, Stachybotrys cartarum, Chephalosporium sp. and Acremonium sp.

The use according to the invention of prochloraz against soft rot can, if required, be widened by adding at least one further microbicidal compound to broaden the activity spectrum or to obtain particular effects. In particular, the activity spectrum may be complemented by adding bactericides and/or termiticides.

Preference is given to using prochloraz in a mixture with one or more of the following termiticidal components:

acetamiprid, allethrin, alpha-cypermethrin, beta-cyfluthrin, bifenthrin, bioallethrin, 4-chloro-2-(2-chloro-2-methylpropyl)-5-[(6-iodo-3-pyridinyl)methoxy]-3(2H)-pyridazinone (CAS-RN: 120955-77-3), chlorfenapyr, chlorpyrifos, clothianidin, cyfluthrin, cyhalothrin, cypermethrin, deltamethrin, etofenprox, fenoxycarb, fipronil, flufenoxuron, hexaflumuron, imidacloprid, nitenpyram, permethrin, pyriproxifen, silafluofen, tebufenozide, thiacloprid, thiamethoxam, tralomethrin, triflumuron.

Particular preference is given to using prochloraz in combination with one or more of the following termiticidal components:

bifenthrin, chlorfenapyr, clothianidin, cyfluthrin, cypermethrin, deltamethrin, etofenprox, imidacloprid, permethrin, thiacloprid, thiamethoxam.

Especially preferred is the use of prochloraz in combination with one or more of the following termiticides:

bifenthrin, clothianidin, imidacloprid, permethrin, thiacloprid.

Preference is also given to using prochloraz in a mixture with one or more of the following bactericidal components:

benzyl alcohol mono-(poly)-hemiformal, ethylene glycol hemiformal, N-(2-hydroxypropyl)amino-methanol, N-methylisothiazolin-3-one, 5-chloro-N-methylisothiazolin-3-one, 4,5-benziso-thiazolinone, formaldehyde, glutaraldehyde, benzalkonium chloride, benzyldimethyltetra-decylammonium chloride, benzyldimethyldodecylammonium chloride, 3-methyl-4-chlorophenol and also 2-benzyl4-chlorophenol and their alkali metal and alkaline earth metal salts, p-hydroxybenzoic esters, and also o-phenylphenol and their alkali metal and alkaline earth metal salts, bronopol, 2,2-dibromo-3-nitrilepropionamide.

Particular preference is given to using prochloraz in combination with one or more of the following bactericides:

benzyl alcohol mono-(poly)-hemiformal, N-methylisothiazolin-3-one, 5-chloro-N-methyliso-thiazolin-3-one, 4,5-benzisothiazolinone, glutaraldehyde, benzalkonium chloride, bronopol, 3-methyl-4-chlorophenol, and also 2-benzyl-4-chlorophenol and the alkali metal and alkaline earth metal salts, o-phenylphenol and their alkali metal and alkaline earth metal salts.

Very particular preference is given to using prochloraz in combination with one or more of the following bactericides:

benzyl alcohol mono-(poly)-hemiformal, 4,5-benzisothiazolinone, benzalkonium chloride, bronopol, 3-methyl-4-chlorophenol, and also 2-benzyl-4-chlorophenol and their sodium and potassium salts, o-phenylphenol and the sodium and potassium salts.

For the use according to the invention, prochloraz can be converted into the customary formulations, such as solutions, emulsions, suspensions, powders, foams, pastes, granules, aerosols and microencapsulations in polymeric substances.

The invention also provides compositions comprising prochloraz for protecting wood and timber products and also wood/plastic composites against attack and/or destruction by soft-rot fungi.

The compositions according to the invention comprise prochloraz and at least one diluent or solvent, if appropriate further auxiliaries and additives and, if appropriate, at least one further microbicidal component, preferably from the group of the bactericides and termiticides mentioned above.

The formulations are prepared in a known manner, for example by mixing the active compound prochloraz and, if appropriate, further active compounds with extenders, i.e. liquid solvents, pressurized liquefied gases and/or solid carriers, if appropriate with the use of surfactants, i.e. emulsifiers and/or dispersants and/or foam formers. If the extender used is water, it is also possible to employ, for example, organic solvents as auxiliary solvents. Suitable liquid solvents are essentially: aromatic compounds, such as xylene, toluene or alkylnaphthalenes, aliphatic hydrocarbons, such as cyclohexane or paraffins, for example mineral oil fractions, alcohols, such as butanol or glycol, and also ethers and esters thereof, ketones, such as acetone, methyl ethyl ketone, methyl isobutyl ketone or cyclohexanone, strongly polar solvents, such as dimethylformamide and dimethyl sulphoxide, and also water. Liquefied gaseous extenders or carriers are to be understood as meaning liquids which are gaseous at ambient temperature and under atmospheric pressure, for example aerosol propellants, such as halogenated hydrocarbons, and also butane, propane, nitrogen and carbon dioxide. Suitable solid carriers are: for example natural ground minerals, such as kaolins, clays, talc, chalk, quartz, attapulgite, montmorillonite or diatomaceous earth, and ground synthetic minerals, such as finely divided silica, alumina and silicates. Suitable solid carriers for granules are: for example crushed and fractionated natural minerals, such as calcite, marble, pumice, sepiolite, dolomite, and also synthetic granules of inorganic and organic meals and also granules of organic material, such as sawdust, coconut shells, corn cobs and tobacco stalks. Suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates, and also protein hydrolysates. Suitable dispersants are: for example lignosulphite waste liquors and methylcellulose.

Tackifiers such as carboxymethylcellulose and natural and synthetic polymers in the form of powders, granules or lattices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian blue, and organic dyes, such as alizarin dyes, azo dyes and metal phthalocyanine dyes, and trace nutrients, such as salts of iron, manganese, boron, copper, cobalt, molybdenum and zinc.

The compositions according to the invention generally comprise between 0.005 and 95% by weight of prochloraz, preferably between 0.1 and 50% by weight of prochloraz, and, if appropriate, between 0.005 and 50% by weight of the microbicidal components mentioned, preferably between 0.1 and 30% by weight.

The microbicidal compositions or concentrates used for protecting the industrial materials comprise the active compound prochloraz or the combination of prochloraz with a further microbicidally active compound in a concentration of from 0.005 to 95% by weight, in particular from 0.1 to 50 per cent by weight.

The application concentrations of the active compound prochloraz to be used according to the invention or of the active compound combination of prochloraz with at least one further microbicidally active compound depends on the nature and the occurrence of the microorganisms to be controlled and on the composition of the material to be protected. The optimum amount to be employed can be determined by test series. In general, the application concentrations of prochloraz or of the combination of prochloraz with at least one further microbicidally active compound are in the range from 0.001 to 5 per cent by weight, preferably from 0.01 to 1.5 per cent by weight, based on the material to be protected.

By using prochloraz according to the invention, it is possible to replace, in an advantageous manner, the microbicidal compositions hitherto available by more effective compositions. The compositions according to the invention have good stability and, in an advantageous manner, a broad activity spectrum.

The present invention furthermore provides, protected against attack and/or destruction by soft-rot fungi, wood and timber products and also wood/plastic composites comprising prochloraz or a composition based on prochloraz.

Wood, timber products and wood/plastic composites which can be protected by the active compound mixtures according to the invention or the compositions comprising them are, by way of example: construction timber, wooden beams, railway sleepers, bridge components, jetties, vehicles made of wood, boxes, pallets, containers, telephone poles, wood lagging, windows and doors made of wood, plywood, medium density fibreboard (MDF), chipboard, oriented strand board (OSB), waferboard, laminated veneer lumber (LVL) or timber products which, quite generally, are used in the construction of houses or building joinery, and also wood/plastic composites.

The invention also provides a method for protecting wood, timber products and wood/plastic composites against attack by soft-rot fungi.

The active compound prochloraz, if appropriate in combination with one or more microbicidally active compounds, can be applied as such, in the form of formulations or the use forms prepared therefrom, such as ready-to-use solutions, suspensions, pastes, soluble powders. Application is in a customary manner by treating the wood, the timber product or the wood/plastic composite with the active compound prochloraz, if appropriate in combination with one or more microbicidally active compounds, or with a preparation prepared therefrom in the form of a formulation or application form, for example by spraying, painting, dipping and industrial impregnation processes, for example vacuum, double vacuum or pressure processes, and by addition to the glue or masterbatch and also via the compounder or mixer.

A particularly effective protection of wood is achieved by industrial impregnation processes, for example by vacuum, double vacuum or pressure processes.

Accordingly, particular preference is given to a method for protecting wood where the wood is impregnated with an effective amount of prochloraz and at least one diluent or solvent, if appropriate further auxiliaries and additives and also, if appropriate, one or more microbicidally active compounds, preferably from the group of the termiticides and bactericides, by vacuum, double vacuum, pressure or dipping processes.

In general, the wood treated as described above, the timber products and the wood/plastic composites comprise between 0.001 and 3% by weight, preferably between 0.002 and 1% by weight and particularly preferably between 0.004 and 0.4% by weight, of prochloraz.

### EXAMPLE

### Activity Against Soft Rot

The activity of prochloraz and of a copper/chromate preparation and tebuconazole, 3-iodo-2-propynyl butylcarbarbamate (IPBC) and imazalil was determined in a test based on ENV 807 (12/2001). To this end, test specimens of the wood type Pinus sylvestris of a size of 40×15×4 mm were vacuum drenched with 0.3 and 0.4% strength active compound solutions and conditioned for 14 days. This was followed by accelerated ageing according to EN 84.

Subsequently, the wood samples were placed on a malt agar medium according to EN 113 and inoculated with a spore suspension according to ENV 807. After 20 weeks of incubation, the reduction in mass of the wood samples treated was determined. The results are compiled in Table 1.

TABLE 1
Test results according to ENV 807

| | | | | | Copper/ | Control |
|---|---|---|---|---|---|---|
| | | | | | chromium | (without |
| Active | Tebuconazole | IPBC | Imazalil | Prochloraz | standard | active |
| compound | (0.3%) | (0.3%) | (0.3%) | (0.3%) | (0.4%) | compound) |
| Reduction in | >4 | >4 | >4 | <2 | <2 | >4 |
| mass in % (mean) | | | | | | |

From Table 1, it is evident that tebuconazole, imazalil and IPBC were insufficiently active against soft-rot fungi, whereas 0.3% prochloraz showed an effect comparable to that of the 0.4% strength copper/chromium standard.

1. Use of prochloraz (N-propy-N-[2-(2,4,6-trichlorophenoxy)ethyl]imidazole-1-carbox-amide) for protecting wood, timber products and wood/plastic composites against attack and/or destruction by soft-rot fungi.

2. Use according to claim 1, characterized in that the material to be protected is treated with prochloraz, if appropriate in the presence of diluents or solvents, auxiliaries and additives.

3. Use according to at least one of claims 1 and 2, characterized in that prochloraz is used in combination with at least one further microbicidally active compound selected from the group of the bactericides and termiticides.

4. Microbicidal composition, comprising prochloraz and at least one diluent or solvent, if appropriate further auxiliaries and additives and, if appropriate, one or more microbicidal components.

5. Composition according to claim 4, comprising from 0.005 to 95% by weight of prochloraz, and, if appropriate, from 0.005 to 50% by weight of one or more microbicidal components selected from the group of the fungicides, insecticides, termiticides and bactericides.

6. Use of a composition according to at least one of claims 4 and 5 for protecting wood, timber products and wood/plastic composites against attack and destruction by soft-rot fungi.

7. Method for protecting wood, timber products and wood/plastic composites, characterized in that the wood, the timber product or the wood/plastic composite is impregnated with prochloraz and at least one diluent or solvent, if appropriate further additives and auxiliaries and, if appropriate, one or more biocidally active compounds by painting, spraying, vacuum, double vacuum, pressure or dip processes and by addition to the glue or masterbatch and also via the compounder.

8. Wood, timber products and wood/plastic composites comprising prochloraz.
